(19) United States
(12) Patent Application Publication
(10) Pub. No.: US 2008/0249515 A1
(43) Pub. Date: Oct. 9, 2008

(54) Interventional Devices and Methods For Laser Ablation

(75) Inventor: Kevin D. Taylor, Colorado Springs, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Spectranetics Corporation, Colorado Springs, CO (US)

(21) Appl. No.: 12/092,819

(22) PCT Filed: Jan. 29, 2007
(86) PCT No.: PCT/US07/02421

§ 371 Date: May 20, 2008

Related U.S. Application Data
(60) Provisional application No. 60/762,972, filed on Jan. 27, 2006.

(51) Int. Cl. A61B 18/20

(52) U.S. Cl. 606/7

(57) ABSTRACT

Laser catheter systems include catheters, mandrels, guidewires, and fiber optics configured to reduce or remove occlusions in a lumen or vessel of a patient. Rotation or translation of a mandrel, a guidewire, or a catheter can induce relative rotational or translational movement between the mandrel or guidewire and the catheter body, and can cause the distal end of the catheter body to rotate or traverse off of a central axis, such as a central longitudinal axis of a proximal or unbent portion of the catheter body, so as to cause ablation energy from the optical fibers to move in an arc or path.

### CROSS-REFERENCES TO RELATED APPLICATIONS

This application claims the benefit of priority from U.S. Patent Application No. 60/762,972 filed Jan. 27, 2006, the entire contents of which are incorporated herein by reference for all purposes.

### BACKGROUND OF THE INVENTION

The embodiments described herein are generally directed to improved devices and methods for the delivery of laser energy within a mammalian subject, including without limitation, to a laser delivery catheter and methods of using same.

Arteries are the primary blood vessels that are responsible for providing blood and oxygen to the heart muscle. Arterial disease occurs when arteries become narrowed or blocked by a buildup of plaque, including atherosclerotic plaque or other deposits. When the blockage is severe, the flow of blood and oxygen to the heart muscle is reduced, causing chest pain. Arterial blockage by clots formed in a human body may be relieved in a number of traditional ways. Drug therapy, including nitrates, beta-blockers, and peripheral vasodilator drugs to dilate the arteries or thrombolytic drugs to dissolve the clot, can be effective in some cases. If drug treatment fails, angioplasty or atherectomy may be used to reform or remove the atherosclerotic plaque or other deposits in the artery. However, often introduction of a balloon in an occluded artery can cause portions of the atherosclerotic material to become dislodged which may cause a total blockage at a point downstream of the subject occlusion thereby requiring emergency procedures. In the event drug therapy is ineffective or other types of angioplasty or atherectomy are either ineffective or too risky, the procedure known as excimer laser atherectomy may be indicated.

In a typical excimer laser atherectomy procedure, a narrow, flexible tube, the laser catheter, is inserted into an artery in the arm or leg. The laser catheter contains one or more optical fibers, which can transmit laser energy. The laser catheter is then advanced inside the artery, sometimes over a previously placed guidewire, to the targeted obstruction at the desired treatment site. After the laser catheter has been positioned, the laser is energized to “remove” the obstruction.

In many procedures, the lesion is often engaged similar to conventional balloon angioplasty by crossing the blockage with a guidewire. The laser catheter's thin, flexible optical fibers facilitate the desired positioning and alignment of the catheter over the guidewire and/or within the vascular system. Using the excimer laser, the clinician performs a controlled blockage removal by sending bursts of ultraviolet light through the catheter and against the blockage, a process called “ablation.” The catheter is then slowly advanced through the blockage reopening the artery. If there are multiple blockages, the catheter is advanced to the next blockage site and the above step is repeated. When the indicated blockages appear to be cleared, the catheter is withdrawn.

However, due to the configuration of the optical fibers in many current laser catheters, the clinician is able to ablate only material that is typically directly in front of the distal end of the catheter. Thus, the debulked tissue area is limited to an area approximately the size of the optical fiber area at the distal end of the catheter. Typically, follow-up atherectomy is recommended. For example, many coronary artery stenoses are located in arteries ranging from 2.0 mm to 4.0 mm diameter. Guide catheters commonly used to access these vessels for atherectomy are about 1.7 mm (6 Fr. Guide) to 2.3 mm (8 Fr. guide) inside diameter. Coronary excimer laser catheters range in tip diameter from 0.9 to 2.0 mm and characteristically ablate tissue equivalent to the area of the catheter tip. For example, a 2.0 mm laser catheter, delivered through a 8 Fr. guide catheter, can ablate a lumen through a stenosis approximately 2 mm in diameter (cross sectional area=3.14 mm²). A 3 mm stenosis has a cross sectional area of 7.1 mm² and a 4.0 mm stenosis has an area of 12.5 mm². Area stenosis reduction for the 2.0 mm catheter is limited to 40% and 25% for the 3.0 mm and 4.0 mm stenoses, respectively. Moreover, many current catheter designs do not provide the clinician with the ability to precisely steer or navigate the laser catheter during an atherectomy procedure.

Thus, it would be desirable to provide improved devices and methods that enable the clinician to ablate or remove a blockage having an area larger than the area of the distal end of the catheter and/or to enhance the clinician's ability to steer or direct the catheter within the vasculature or other target area in the patient's body.

### BRIEF SUMMARY OF THE INVENTION

In accordance with some embodiments, without limitation, the invention comprises devices and methods that meet these unmet needs. Embodiments of the present invention, for example, include laser catheter systems having catheters, mandrels, guidewires, and fiber optics configured to reduce or remove occlusions in a lumen or vessel of a patient. Rotation or translation of a mandrel, a guidewire, or a catheter can induce relative rotational or translational movement between the mandrel or guidewire and the catheter body, and can cause the distal end of the catheter body to rotate or traverse off of a central axis, such as a central longitudinal axis of a proximal or unbent portion of the catheter body, so as to cause ablation energy from the optical fibers to move in an arc or path.

In a first aspect, embodiments of the present invention provide a laser catheter system. The system includes, for example, a laser catheter comprising a catheter body having a proximal end, a distal end, a central axis, and a mandrel lumen that is generally aligned with the central axis. The laser catheter further includes a plurality of optical fibers extending to the distal end, and a mandrel having a proximal end and a distal end. The mandrel includes a bend near the distal end. The mandrel is insertable into the mandrel lumen, with the proximal end of the mandrel extending beyond the proximal end of the laser catheter, and the bend of the mandrel being near the distal end of the catheter body such that rotation of the mandrel from the proximal end of the mandrel causes the distal end of the catheter body to rotate off of the central axis so as to cause the laser energy from the optical fibers to move in an arc. The catheter system may also include a laser system for supplying laser energy to the fiber optics. The mandrel may include or may be a guidewire. In some cases, the optical fibers surround the mandrel lumen and the catheter body includes a jacket surrounding the optical fibers. A bend in the mandrel can be within about 0.5 cm to about 2.5 cm of the distal end of the mandrel. The distal end of the catheter body can have a diameter that is in the range from about 0.5 mm to about 2.5 mm, and the bend in the mandrel can permit the laser energy to reach an area that is at least about 2 times the diameter of the distal end of the catheter body. In some cases, the bend has an angle relative to the central axis that is in the range from about 1 degree to about 89 degrees. The catheter may also have a guidewire lumen extending between the proximal end and the distal end, and further include a guidewire that is insertable through the guidewire lumen. In some cases, the mandrel includes a plurality of bends near the distal end. Optionally, the mandrel has a diameter near the distal end that is in the range from about 0.1 mm to about 0.5 mm, and the distal end is formed in the shape of a ball.

In another aspect, embodiments of the present invention encompass a laser catheter system than includes a laser catheter having a catheter body with a proximal end, a distal end, a central axis, and a mandrel lumen that is generally aligned with the central axis. The mandrel lumen can have a size or diameter in the range from about 0.2 mm to about 0.7 mm, and the laser catheter can further include a plurality of optical fibers extending to the distal end. The distal end of the catheter body can have a diameter that is in the range from about 0.5 mm to about 2.5 mm. The laser catheter system may also include a mandrel having a proximal end and a distal end. The mandrel may include a bend near the distal end. The mandrel is insertable into the mandrel lumen, with the proximal end of the mandrel extending beyond the proximal end of the laser catheter, and the bend of the mandrel being near the distal end of the catheter body such that rotation or movement of the mandrel from the proximal end of the mandrel causes the distal end of the catheter body to rotate or move off of the central axis. Optionally, the mandrel includes or is a guidewire. In some cases, the bend of the mandrel is near the distal end of the catheter body such that rotation of the mandrel from the proximal end of the mandrel causes the distal end of the catheter body to rotate off of the central axis. In some cases, movement of the distal end of the catheter body off of the central axis causes the laser energy from the optical fibers to move in a path that ablates an area that is at least about 2 times the diameter of the distal end of the catheter body.

In another aspect, embodiments of the present invention encompass methods for treating a region in a vessel. In an exemplary embodiment, a method includes inserting a laser catheter into a vessel, where the laser catheter includes a catheter body having a proximal end, a distal end, a distal tip at the distal end, a central axis, and a mandrel lumen that is generally aligned with the central axis. The laser catheter includes a plurality of optical fibers extending to the distal end. The method can also include inserting a mandrel into the mandrel lumen, wherein the mandrel has a distal end, a proximal end and a bend near the distal end. The mandrel is inserted until the bend is near the distal end of the catheter body. The method may also include rotating the mandrel to place the distal tip of the catheter body at a certain location within the vessel which is offset from the central axis. Further, the method may include providing laser energy to the optical fibers to permit laser energy to project from the distal tip at the certain location. The method may also include continuously rotating the mandrel to sweep the laser energy in an arc within the vessel. Optionally, the method may include coupling the laser catheter to a laser system to supplying laser energy to the fiber optics. In some cases, the optical fibers surround the mandrel lumen, the catheter body includes a jacket surrounding the optical fibers, and the mandrel is inserted between the optical fibers. The mandrel may be inserted through the catheter body until the bend in the mandrel is within about 0 cm to about 5 cm of the distal end of the catheter body. The mandrel may include or may be a guidewire. The distal end of the catheter body can have a diameter that is in the range from about 0.6 mm to about 2.5 mm, and laser energy can be swept to ablate an area that is larger than or at least about 2 times the diameter of the distal end of the catheter body. In some cases, the bend has an angle relative to the central axis that is in the range from about 1 degree to about 89 degrees. In some embodiments, the method may also include introducing a guidewire into the vessel, inserting the laser catheter over the guidewire using the mandrel lumen to situate the laser catheter within the vessel, and removing the guidewire prior to introducing the mandrel. The catheter body may also include a guidewire lumen extending between the proximal end and the distal end, and the method may also encompass inserting a guidewire through the guidewire lumen and introducing the laser catheter into the vessel using the guidewire. The mandrel can include a pair of bends, and the mandrel can be inserted through the catheter body such that the first bend extends beyond the distal tip and the second bend is at the distal tip. In some cases, the mandrel includes a plurality of bends near the distal end and the method includes applying laser energy to the optical fibers while distally advancing the laser catheter over the plurality of bends. Optionally, the mandrel may include or may be a guidewire.

### BRIEF DESCRIPTION OF THE DRAWINGS

FIGS. 1A-1D illustrate a laser catheter system according to embodiments of the present invention.

FIGS. 2A-2D depict a laser catheter system according to embodiments of the present invention.

FIGS. 3A-3D show a laser catheter system according to embodiments of the present invention.

FIGS. 4A-4B illustrate a laser catheter system according to embodiments of the present invention.

FIGS. 5A-5D illustrate laser catheter systems according to embodiments of the present invention.

FIGS. 6A-6B depict aspects a laser catheter system and method according to embodiments of the present invention.

### DETAILED DESCRIPTION OF THE INVENTION

Without limiting the scope of the invention to only the embodiments described herein, the present invention comprises a laser catheter which fits within a small guide catheter yet completely ablates a stenosis up to 4.0 mm in diameter, as well as a laser catheter which can clear a stenosis up to 2 times (or more) the diameter of the catheter tip. Embodiments encompass catheter systems that provide a moveable catheter tip, whereby the catheter tip can be deflected or swept over an area that is greater than the diameter of the catheter tip. In some embodiments, without limitation, the invention also comprises an improved device to steer the catheter within the patient's body, including without limitation, in the vasculature. The invention also comprises methods of using same to accomplish ablation and other medical interventions using laser energy.

In some embodiments, without limitation, the invention comprises a laser catheter with a bent mandrel or guidewire that is or becomes inserted therein. One or more bends in the wire or mandrel are located near the distal tip of the laser catheter to produce deflection or offset of the catheter tip. When the wire or mandrel is rotated or translated, for example relative to the catheter body, the catheter tip sweeps in a circular and/or offset manner. The degree of sweep can be adjusted by varying the degree of a bend on the guidewire or mandrel. The present techniques are well suited for use in directing a distal tip of a laser catheter toward or at an occlusion within a vessel. For example, the distal tip of a laser catheter can be laterally offset from a first position to a second position, where the general alignment of the catheter body relative to the vessel is not substantially changed. In some cases, the distal tip of the catheter is deflected or offset in a steering method when advancing the catheter within the vessel. Often, an ablation procedure is performed via a distal tip of the catheter, and the catheter tip or body is moved longitudinally relative to the vessel during the ablation.

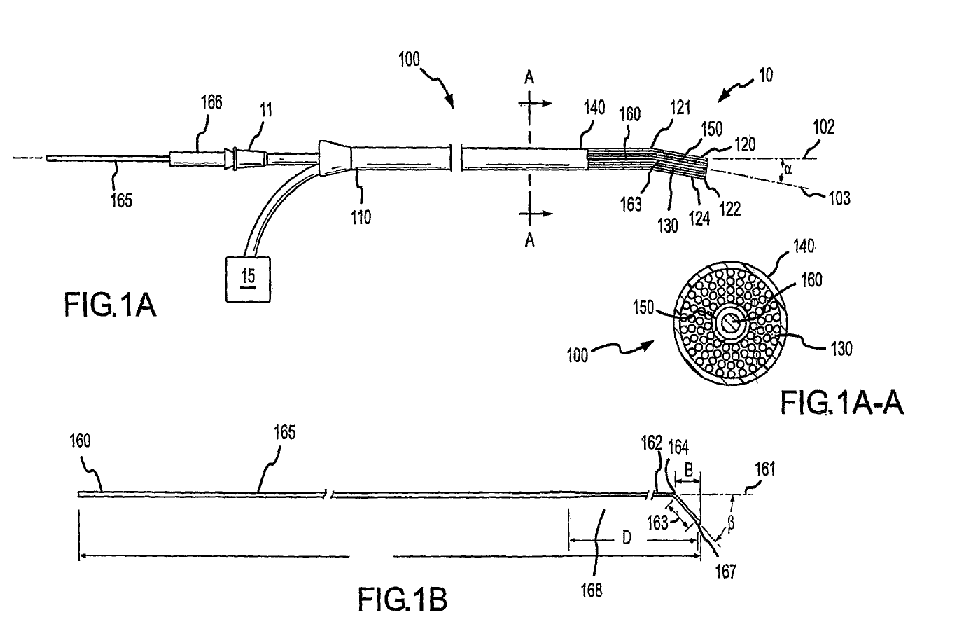

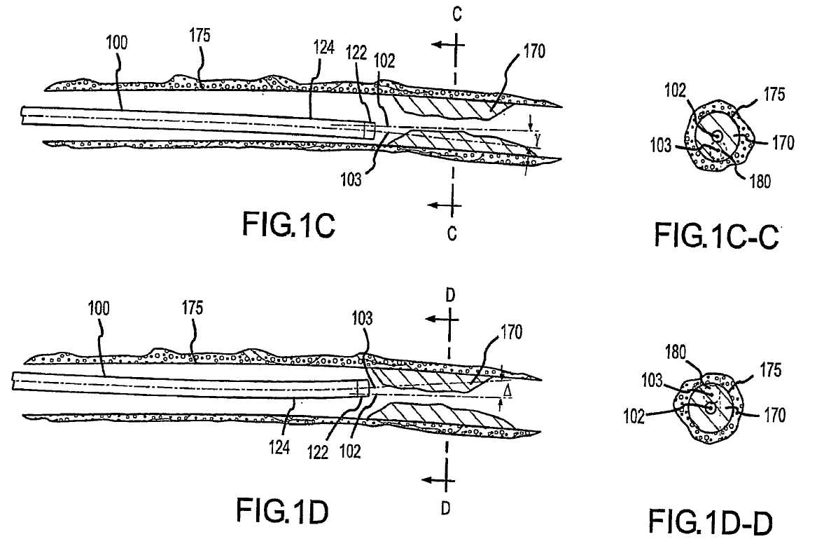

Turning now to the drawings, in a first described embodiment as depicted in FIGS. 1A-1D, a laser catheter system 10 comprises a laser catheter or catheter body 100 with a more proximal section or end 110 and a more distal section or end 120. In some cases, the diameter of the distal section or end 120 is within a range from about 0.5 mm to about 2.5 mm. As shown in FIG. 1A, catheter body 100 can have or define a central longitudinal axis 102. Laser catheter system 10 can also include a mandrel 160, and a torque handle 166 for rotating the mandrel 160 relative to the catheter 100. The laser catheter system 10 may further include a proximal guidewire port 11 that is adapted to the receive mandrel 160 or a guidewire, or both. The catheter 100 is comprised of, or is adapted to house, a plurality of optical fibers 130 for transmission of laser energy. The optical fibers 130 are disposed within the catheter 100, surrounded by an outer jacket 140 of the catheter body, and extended toward distal section 120 or distal tip 122, as shown in cross-section 1A-A. The optical fibers 130 are disposed around or surround a lumen 150 inside of the catheter 100. Mandrel lumen 150 can have a size or diameter that is within a range from about 0.2 mm to about 0.6 mm. Catheter system 10 may also include or be coupled with a laser system 15 for supplying laser energy to the fiber optics 130.

As noted above, the laser catheter system 10 can also comprise a mandrel 160, which may include a proximal end or section 165 and a distal end or section 162. Mandrel 160 can include one or more bends near the distal end of the mandrel, and can be inserted into the mandrel lumen 150. For example, mandrel 160 can be bent in a more distal segment or end 162, so as to form a bend 164, as depicted in FIG. 1B. The distal tip 167 of mandrel 160 may be formed in any desired shape. For example, distal tip 167 may be formed in the shape of a ball. In some cases, all or a portion of the mandrel may include a radiopaque material. Mandrel 160 may have a total length L along axis 161 that is within a range from about 100 cm to about 170 cm. Mandrel 160 may also have a tapered distal part 168 that spans or extends a distance D along axis 161 that is within a range from about 10 cm to about 40 cm. In some cases, the bent segment 163 spans or extends a distance B along axis 161 that is within a range from about 0.1 cm to about 1.0 cm. A distal tip ball may include a radiopaque material. The length of the bent segment 163 may be varied as desired. For example, bent segment 163 can have a length within a range from about 0.1 cm to about 4 cm. In some embodiments, the distance between bend 164 and the distal tip or end 167 of the mandrel is within a range from about 0.5 cm to about 2.5 cm. Bend 164 of mandrel distal segment 162 can provide an angle β between a central longitudinal axis 161 of the mandrel proximal portion 165 and a central longitudinal axis of bent segment 163. The angle β of the bend 164 may be varied from between about 1 and about 89 degrees, with about 45 degrees comprising one embodiment. Angle β can in some cases be defined as the angle between the bent segment 163 and the longitudinal axis 161 that corresponds to the more proximal segment 165 of mandrel 160.

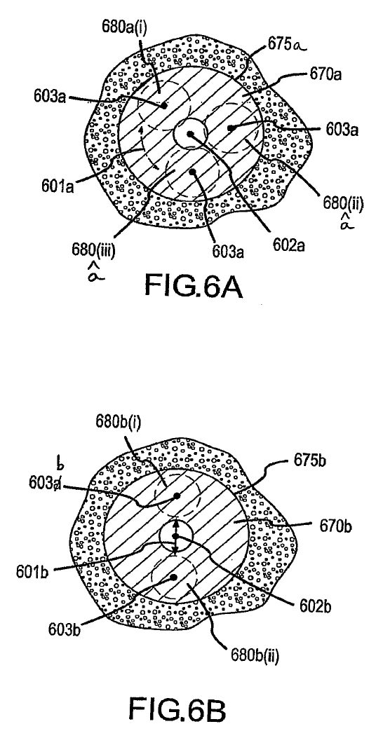

Mandrel 160 can be configured to induce or contribute to a bend 121 in the catheter body, as depicted in FIG. 1A. Thus, a proximal portion of mandrel lumen 150 can be generally aligned with or substantially parallel to central axis 102 of the proximal portion of catheter body 100, and the distal portion of mandrel lumen 150 can be generally aligned with or substantially parallel to a central axis 103 of the distal portion of catheter body 100. In some embodiments, the mandrel 160 is insertable into the mandrel lumen 150, with the proximal end 165 of the mandrel extending beyond the proximal end 110 of the laser catheter. When the catheter 100 is disposed over the mandrel 160, or when mandrel 160 is inserted or into catheter 100, the bent mandrel 160 produces a bend 121 in the catheter distal section 122. Bend 121 can be associated with or defined by an angle α between the central longitudinal axis 102 of the catheter body 100 and a central longitudinal axis 103 of the deflected end segment or portion 124 of the catheter body. Angle α of bend 121 may be varied from between about 1 degree and about 89 degrees, with about 45 degrees comprising one embodiment. When the mandrel 160 is rotated, it produces circular deflection of the tip 122 of the catheter 100. Rotation of the mandrel 160 from the proximal end 165 of the mandrel, for example by a torque handle 166, can induce relative rotational movement between the mandrel 160 and the catheter body 100, and thus cause the distal end 122 of the catheter body 100 to rotate off of the central axis 102 at an angle α so as to cause the laser energy from the optical fibers 130 to move in an arc, as further illustrated in FIG. 6A. Angle α will typically be less than angle β. This may be due to the comparative stiffness of the catheter and the mandrel. For example, the catheter is often relatively stiff as compared with a thin and more flexible mandrel, and less than 100% of the mandrel bend is imparted to the catheter. The bend 164 in the mandrel 160 can permit the laser energy to reach an area that is at least about 2 times the diameter of the distal end 122 of the catheter body 100. As seen in FIG. 1B, the distal segment 162 of mandrel may have a diameter that is smaller than the diameter of the more proximal segment 165 of the mandrel. In some cases, the diameter of the distal segment 162 or the distal end or tip 167 is within a range from about 0.2 mm to about 0.5 mm. A diameter of a distal ball can be within a range from about 0.2 mm to about 0.6 mm. A diameter of a mandrel just proximal to a distal ball can be within a range from about 0.1 mm to about 0.5 mm.

In use, the catheter 100 can be positioned in a subject, for example by insertion over a previously placed guidewire (not shown in FIGS. 1A-1D) or otherwise, in proximity to a stenosis or occlusion 170 in a vascular wall 175, as depicted in FIGS. 1C and 1D. The guidewire is removed, and/or the bent mandrel 160 is inserted into the lumen 150 of the catheter 100 such that the bent segment 163 of the mandrel 160 is disposed within the more distal section 120 of the catheter. In some embodiments, the proximal end 165 of the mandrel 160 may extend proximally beyond the proximal end 110 of the laser catheter 100. In some cases, mandrel 160 may include or be a guidewire. Laser energy is applied according to methods known to those of ordinary skill in the art. In accordance with some embodiments, during application of laser energy, the mandrel 160 is rotated such that the laser energy is directed at an angle γ from the longitudinal axis 102 of the unbent section or more proximal segment 110 of the catheter, as depicted in FIG. 1C, or at an angle Δ from the longitudinal axis 102 of the unbent section or more proximal segment 110 of the catheter, as depicted in FIG. 1D, thus “sweeping” the occlusion with laser energy over an area that is greater that the surface area of the distal end of the catheter. This placement of the laser energy is further illustrated in FIG. 1C-C, where the catheter tip position 180 is shown as slightly below of offset from the central axis 102 of the catheter body, and in FIG. 1D-D, where the catheter tip position 180 is shown as slightly below or offset from the central axis 102 of the catheter body. In some embodiments, a central longitudinal axis of the laser energy can correspond to the central longitudinal axis 103 of the deflected end segment or portion 124 of the catheter body. A bend 164 of a mandrel 160 can be disposed near the distal end 120 of the catheter body 100 such that rotation of mandrel 160 from the proximal end 165 of the mandrel 160 causes the distal end 120 of the catheter body to rotate off of the central axis 102 so as to cause the laser energy from the optical fibers to move in an arc that sweeps an area that is at least about 2 times the diameter of the distal end 120 of the catheter body 100.

Some embodiments of the present invention encompass a method for treating a region in a vessel. The method can include inserting a laser catheter 100 into a vessel 175. The laser catheter 100 can have a proximal end 110, a distal end 120, a central axis 102 which can correspond with the proximal end, and a mandrel lumen 150. The mandrel lumen 150 can be generally aligned with the central axis 102. The laser catheter 100 can also include a plurality of optical fibers 130 extending to the distal end 120. The method also includes inserting a mandrel 160 into the mandrel lumen 150. The mandrel 160 can have a distal end 162, a proximal end 165, and a bend 164 near the distal end 162. The mandrel 160 can be advanced distally or otherwise inserted into the mandrel lumen 150 until the bend 164 is at or near the distal end 120 of the catheter body 100. The method further includes rotating the mandrel 160 to place the distal tip 122 of the catheter body 100 at a certain location 180 within the vessel 175, where the location 180 is offset from the central axis 102 of the catheter body. Additionally, the method includes providing laser energy to the optical fibers 130 to permit laser energy to project from the distal tip 122 of the catheter body 100 at the certain location 180. In some embodiments, the method includes continuously rotating the mandrel 160 to sweep the laser energy in an arc within the vessel. Optionally, the method may include inducing deflection in the distal end 120 of the catheter body 100 by advancing the mandrel 160 into the mandrel lumen 150 or retracting the mandrel 160 proximally therefrom. Relatedly, deflection of the distal tip 122 of the catheter body 100 can be achieved by longitudinally translating the catheter body 100 relative to the mandrel 160, or by longitudinally translating the mandrel 160 relative to the catheter body 100, or both. The method may also include coupling the laser catheter 100 to a laser system 15 to supplying laser energy to the fiber optics 130. The optical fibers 130 can surround the mandrel lumen 150. The catheter body 100 can include a jacket 140 surrounding the optical fibers 130. The mandrel 160 can be inserted between the optical fibers 130. In some embodiments, the mandrel 160 is inserted through the catheter body 100 until the bend 164 in the mandrel 160 is within about 0 cm to about 5 cm of the distal end or tip 122 of the catheter body 100. The mandrel 160 can include or can be a guidewire. The distal end 120 of the catheter body can have a diameter that is in the range from about 0.6 mm to about 2.5 mm, and the laser energy can be swept to ablate an area that is at least about 2 times the diameter of the distal end of the catheter body. The bend 164 in the mandrel 160 can have an angle relative to the central axis 102 of the catheter body 100 that in the range from about 1 degree to about 89 degrees. Optionally, a method embodiment may include introducing a guidewire into the vessel 175, inserting the laser catheter 100 over the guidewire using the mandrel lumen 150 to situate the laser catheter 100 within the vessel 175 and removing the guidewire prior to introducing the mandrel 160. The catheter body 100 may further include a guidewire lumen extending between the proximal end 110 and the distal end 120, and the method may include inserting a guidewire through the guidewire lumen and introducing the laser catheter 100 into the vessel 175 using the guidewire. In some cases, the mandrel 160 includes a pair of bends, and the method includes inserting the mandrel 160 through the catheter body 100 such that the first bend extends beyond the distal tip 122 and the second bend is at the distal tip 122. In some cases, the mandrel 160 includes a plurality of bends near the distal end 120, and the method includes apply laser energy to the optical fibers 130 while distally advancing the laser catheter 100 over the plurality of bends. Optionally, the mandrel can include or can be a guidewire.

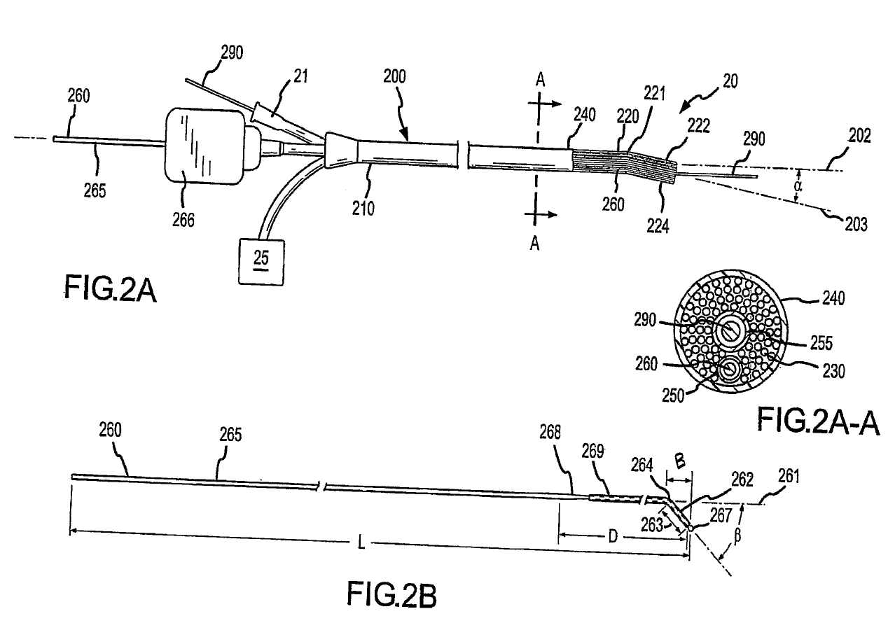

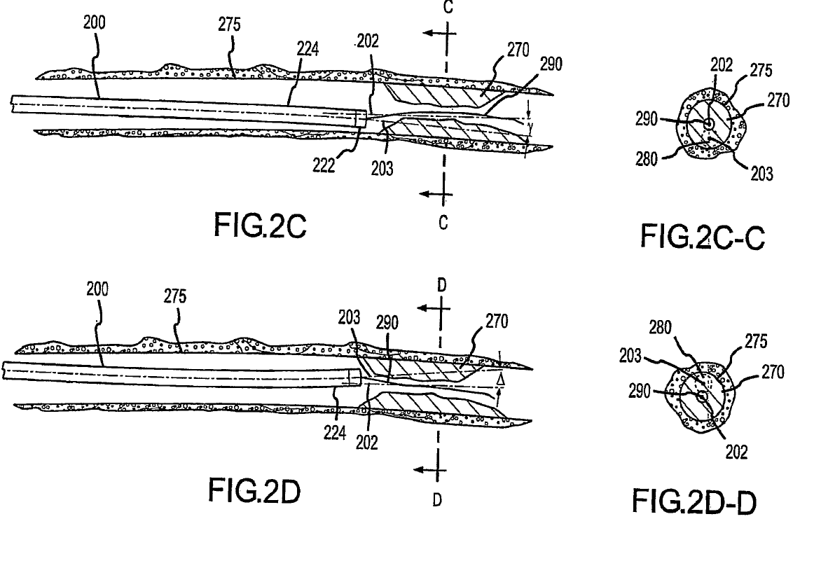

A further described laser catheter system 20 embodiment shown in FIGS. 2A-2D can include elements from any embodiment described herein, and in some cases the catheter 200 comprises at least two lumens. For example, catheter body 200 can include a mandrel lumen 250 and a guidewire lumen 255, such that the bent mandrel 260 resides in its own lumen within the catheter or catheter body 200. This allows use of a guidewire 290 during lasing and for positioning of the mandrel 260 to vary the stiffness of the distal catheter end 220 for navigation into the vascular anatomy 275. In some embodiments, the guidewire 290 optionally further extends from the distal tip 222 of the catheter 200 so as to penetrate or cross a stenosis or occlusion 270. The laser catheter system 20 can also include a motorized device 266 that can engage the mandrel 260. For example, the motorized device 266 can include a torque component that rotates or applies torque to the mandrel 260. Optionally, the motorized device 266 can include a translation component that advances the mandrel 260 distally or retracts the mandrel 260 proximally, or both. As illustrated in FIG. 2A, the catheter body 200 can include a guidewire lumen 255 that extends between the catheter proximal end 210 and the catheter distal end 220. The catheter 200 can also include a guidewire 290 that is insertable through the guidewire lumen 255.

As shown in FIG. 2A, catheter body 200 can have or define a central longitudinal axis 202. Laser catheter system 20 can also include a mandrel 260, and an automated torque device 266 for rotating the mandrel 260 relative to the catheter 200. The laser catheter system 20 may further include a proximal guidewire port 21 that is adapted to the receive mandrel 260 or a guidewire 290, or both. The catheter 200 is comprised of, or is adapted to house, a plurality of optical fibers 230 for transmission of laser energy. The optical fibers 230 are disposed within the catheter 200, surrounded by an outer jacket 240 of the catheter body, and extended toward distal section 220 or distal tip 222, as shown in cross-section 2A-A. Guidewire lumen 255 can be disposed in or toward a central region of the cross-section, and mandrel lumen 250 can be disposed in or toward a peripheral region of the cross-section. In some embodiments, the guidewire lumen 255 may be disposed more peripherally, and the mandrel lumen may be disposed more centrally. The optical fibers 230 are disposed at least partially around a mandrel lumen 250 and a guidewire lumen 255 inside of the catheter 200. Mandrel lumen 250 can have a size or diameter that is within a range from about 0.2 mm to about 0.6 mm. Catheter system 20 may also include or be coupled with a laser system 25 for supplying laser energy to the fiber optics 230.

As noted above, the laser catheter system 20 can also comprise a mandrel 260, which may include a proximal end or section 265 and a distal end or section 262. Mandrel 260 can include one or more bends near the distal end of the mandrel, and can be inserted into the mandrel lumen 250. For example, mandrel 260 can be bent in a more distal segment or end 262, so as to form a bend 264, as depicted in FIG. 2B. The distal tip 267 of mandrel 260 may be formed in any desired shape. For example, distal tip 267 may be formed in the shape of a ball. In some cases, all or a portion of the mandrel may include a radiopaque material. Mandrel 260 may have a total length L along axis 261 that is within a range from about 100 cm to about 170 cm. Mandrel 260 may also have a tapered distal part 268 that spans or extends a distance D along axis 261 that is within a range from about 10 cm to about 40 cm. The system 20 may include a sleeve 269 disposed at least partially around mandrel 260. For example, system 20 may include a sleeve 269 that contains PTFE and is disposed about a tapered section 268 of the mandrel 260. In some cases, the bent segment 263 spans or extends a distance B along axis 261 that is within a range from about 0.1 cm to about 1.0 cm. A distal tip ball may include a radiopaque material. The length of the bent segment 263 may be varied as desired. For example, bent segment 263 can have a length within a range from about 0.1 cm to about 4 cm. In some embodiments, the distance between bend 264 and the distal tip or end 267 of the mandrel is within a range from about 0.5 cm to about 2.5 cm. Bend 264 of mandrel distal segment 262 can provide an angle β between a central longitudinal axis 261 of the mandrel proximal portion 265 and a central longitudinal axis of bent segment 263. The angle β of the bend 264 may be varied from between about 1 and about 89 degrees, with about 45 degrees comprising one embodiment. Angle β can in some cases be defined as the angle between the bent segment 263 and the longitudinal axis 261 that corresponds to the more proximal segment 265 of mandrel 260.

Mandrel 260 can be configured to induce or contribute to a bend 221 in the catheter body, as depicted in FIG. 2A. Thus, a proximal portion of mandrel lumen 250 can be generally aligned with or substantially parallel to central axis 202 of the proximal portion of catheter body 200, and the distal portion of mandrel lumen 250 can be generally aligned with or substantially parallel to a central axis 203 of the distal portion of catheter body 200. In some embodiments, the mandrel 260 is insertable into the mandrel lumen 250, with the proximal end 265 of the mandrel extending beyond the proximal end 210 of the laser catheter. When the catheter 200 is disposed over the mandrel 260, or when mandrel 260 is inserted or into catheter 200, the bent mandrel 260 produces a bend 221 in the catheter distal section 222. Bend 221 can be associated with or defined by an angle α between the central longitudinal axis 202 of the catheter body 200 and a central longitudinal axis 203 of the deflected end segment or portion 224 of the catheter body. Angle α of bend 221 may be varied from between about 1 degree and about 89 degrees, with about 45 degrees comprising one embodiment. When the mandrel 260 is rotated, it produces circular or offset deflection of the tip 222 of the catheter 200. Rotation of the mandrel 260 from the proximal end 265 of the mandrel, for example by a motorized torque device 266, can induce relative rotational movement between the mandrel 260 and the catheter body 200, and thus cause the distal end 222 of the catheter body 200 to rotate off of the central axis 202 at an angle α so as to cause the laser energy from the optical fibers 230 to move in an arc, as further illustrated in FIG. 6A. Accordingly, in some embodiments angle α corresponds with angle β. The bend 264 in the mandrel 260 can permit the laser energy to reach an area that is at least about 2 times the diameter of the distal end 222 of the catheter body 200. As seen in FIG. 2B, the distal segment 262 of mandrel may have a diameter that is smaller than the diameter of the more proximal segment 265 of the mandrel. In some cases, the diameter of the distal segment 262 or the distal end or tip 267 is within a range from about 0.1 mm to about 0.5 mm.

In use, the catheter 200 can be positioned in a subject, for example by insertion over a previously placed guidewire 290 or otherwise, in proximity to a stenosis or occlusion 270 in a vascular wall 275, as depicted in FIGS. 2C and 2D. The guidewire is removed, or left in place, and/or the bent mandrel 260 is inserted into the lumen 250 of the catheter 200 such that the bent segment 263 of the mandrel 260 is disposed within the more distal section 220 of the catheter. In some embodiments, the proximal end 265 of the mandrel 260 may extend proximally beyond the proximal end 210 of the laser catheter 200. In some cases, mandrel 260 may include or be a guidewire. Laser energy is applied according to methods known to those of ordinary skill in the art. In accordance with some embodiments, during application of laser energy, the mandrel 260 is rotated such that the laser energy is directed at an angle γ from the longitudinal axis 202 of the unbent section or more proximal segment 210 of the catheter, as depicted in FIG. 2C, or at an angle Δ from the longitudinal axis 202 of the unbent section or more proximal segment 210 of the catheter, as depicted in FIG. 2D, thus “sweeping” the occlusion with laser energy over an area that is greater that the surface area of the distal end of the catheter. This placement of the laser energy is further illustrated in FIG. 2C-C, where the catheter tip position 280 is shown as slightly below of offset from the central axis 202 of the catheter body, and in FIG. 2D-D, where the catheter tip position 280 is shown as slightly below or offset from the central axis 202 of the catheter body. In some embodiments, a central longitudinal axis of the laser energy can correspond to the central longitudinal axis 203 of the deflected end segment or portion 224 of the catheter body. A bend 264 of a mandrel 260 can be disposed near the distal end 220 of the catheter body 200 such that rotation of mandrel 260 from the proximal end 265 of the mandrel 260 causes the distal end 220 of the catheter body to rotate off of the central axis 202 so as to cause the laser energy from the optical fibers to move in an arc that sweeps an area that is at least about 2 times the diameter of the distal end 220 of the catheter body 200.

Some embodiments of the present invention encompass a method for treating a region in a vessel. The method can include inserting a laser catheter 200 into a vessel 275. The laser catheter 200 can have a proximal end 210, a distal end 220, a central axis 202 which can correspond with the proximal end, and a mandrel lumen 250. The mandrel lumen 250 can be generally aligned with or parallel to the central axis 202. The catheter can also have a guidewire lumen 255 that is generally aligned with or parallel to the central axis 202. The laser catheter 200 can also include a plurality of optical fibers 230 extending to the distal end 220. The method includes inserting a mandrel 260 into the mandrel lumen 250, and may also include inserting a guidewire 290 into the guidewire lumen 255, and advancing, retracting, or otherwise translating the catheter along the guidewire 290. The mandrel 260 can have a distal end 262, a proximal end 265, and a bend 264 near the distal end 262. The mandrel 260 can be advanced distally or otherwise inserted into the mandrel lumen 250 until the bend 264 is at or near the distal end 220 of the catheter body 200. The method further includes rotating the mandrel 260 to place the distal tip 222 of the catheter body 200 at a certain location 280 within the vessel 275, where the location 280 is offset from the central axis 202 of the catheter body. Additionally, the method includes providing laser energy to the optical fibers 230 to permit laser energy to project from the distal tip 222 of the catheter body 200 at the certain location 280. In some embodiments, the method includes continuously rotating the mandrel 260 to sweep the laser energy in an arc within the vessel. Optionally, the method may include inducing deflection in the distal end 220 of the catheter body 200 by advancing the mandrel 260 into the mandrel lumen 250 or retracting the mandrel 260 proximally therefrom. Relatedly, deflection of the distal tip 222 of the catheter body 200 can be achieved by longitudinally translating the catheter body 200 relative to the mandrel 260, or by longitudinally translating the mandrel 260 relative to the catheter body 200, or both. The method may also include coupling the laser catheter 200 to a laser system 25 to supplying laser energy to the fiber optics 230. The optical fibers 230 can surround the mandrel lumen 250 and the guidewire lumen 255. In some cases, the optical fibers 230 partially surround the mandrel lumen 250, the guidewire lumen 255, or both. The catheter body 200 can include a jacket 240 surrounding the optical fibers 230. The mandrel 260 and guidewire 290 can be inserted between the optical fibers 230. In some embodiments, the mandrel 260 is inserted through the catheter body 200 until the bend 264 in the mandrel 260 is within about 0 cm to about 5 cm of the distal end or tip 222 of the catheter body 200. The mandrel 260 can include or can be a guidewire. The distal end 220 of the catheter body can have a diameter that is in the range from about 0.6 mm to about 2.5 mm, and the laser energy can be swept across an area that is at least about 2 times the diameter of the distal end of the catheter body. The bend 264 in the mandrel 260 can have an angle relative to the central axis 202 of the catheter body 200 that in the range from about 1 degree to about 89 degrees. Optionally, a method embodiment may include introducing a guidewire into the vessel 275, inserting the laser catheter 200 over the guidewire 290 using the guidewire lumen 255 to situate the laser catheter 200 within the vessel 275 and removing the guidewire 290 prior to introducing the mandrel 260, or optionally leaving the guidewire 290 in place. The catheter body 200 may further include a guidewire lumen 255 extending between the proximal end 210 and the distal end 220, and the method may include inserting a guidewire 290 through the guidewire lumen 255 and introducing the laser catheter 200 into the vessel 275 using the guidewire 290. In some cases, the mandrel 260 includes a pair of bends, and the method includes inserting the mandrel 260 through the catheter body 200 such that the first bend extends beyond the distal tip 222 and the second bend is at the distal tip 222. In some cases, the mandrel 260 includes a plurality of bends near the distal end 220, and the method includes apply laser energy to the optical fibers 230 while distally advancing the laser catheter 200 over the plurality of bends. Optionally, the mandrel can include or can be a guidewire.

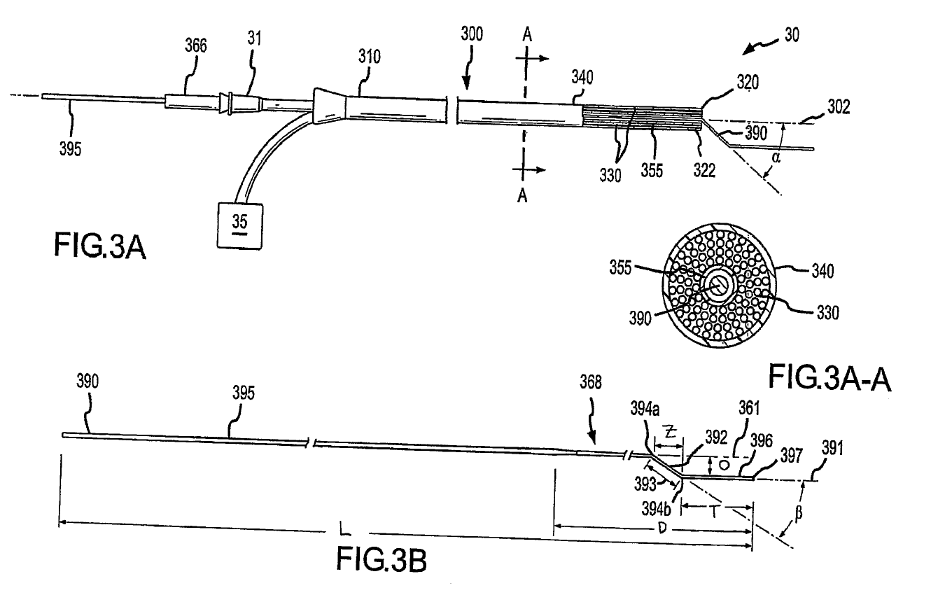

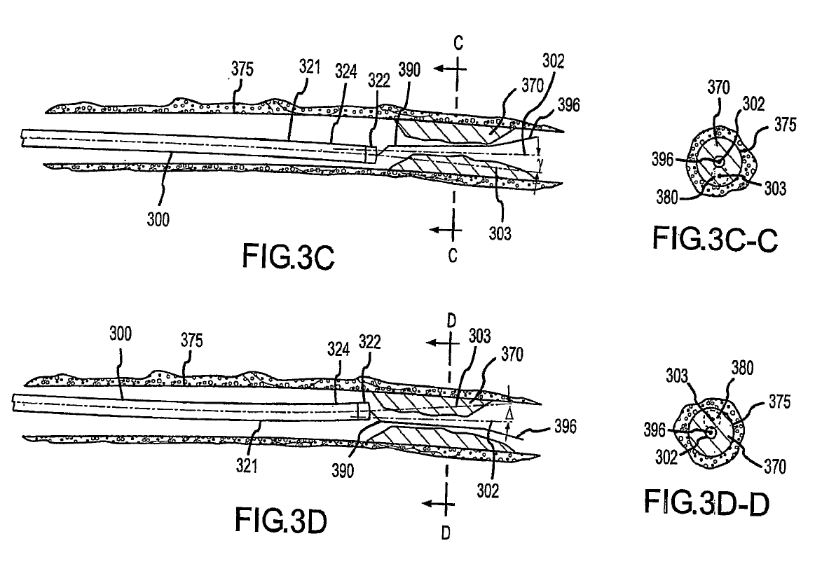

In another described embodiment as depicted in FIGS. 3A-3D, a laser catheter system 30 comprises a laser catheter or catheter body 300 with a more proximal section 310 and a more distal section 320. The catheter 300 is comprised of a plurality of optical fibers 330 for transmission of laser energy that are disposed within the catheter 300 and surrounded by an outer jacket 340. The optical fibers 330 are disposed around a lumen 355 inside of the catheter 300. Without limiting the scope of the invention, the laser catheter system 30 also comprises a guidewire 390 that is bent in a more distal segment 392. In some embodiments, at about 3 cm to about 20 cm from a distal end or tip 397 of the guidewire 390, the guidewire 390 is bent at about a 30 degree offset at a more distal bend 394b, followed by another opposing more proximal bend 394a on the guidewire 390. The segment 393 of guidewire 390 between bends 394a and 394b can be any desired length, and can be considered to span a length Z along a central longitudinal axis 361 defined by the proximal section 395 of guidewire 390. The bends 394a, 394b are such that in use the distal tip 397 of the guidewire 390 can be offset from the longitudinal axis 302 of the catheter 300 a distance O which can be within a range from about 0.5 to about 6 mm when the catheter 300 is disposed over a more proximal portion 395 of the guidewire 390, as shown in FIG. 3B. The degree to which the distal tip of the catheter can be deflected or offset during use is often related to the geometrical configuration of the distal end of the guidewire. For example, a guidewire having a larger distance O may be well suited for imparting larger deflections or offsets in the distal tip of the catheter. When the catheter 300 is positioned in a subject in proximity to a stenosis or occlusion 370, the distal end 396 of the guidewire 390 is placed so as to penetrate or cross the occlusion 370. Laser energy may be applied according to methods known to those of ordinary skill in the art. In accordance with embodiments of the present invention, during application of laser energy, rotation of the bent guidewire 390 will produce deflection of the catheter tip 322 in a circular path, allowing the tip 322 to cover and ablate an area much larger than the diameter of the tip 322. The distal section 396 of the guidewire 390 can act as a strut to help obtain deflection of the catheter tip 322 within the artery. In addition, in some embodiments, without limitation, deflection of the tip 322 by one or more bends (e.g. bends 394a, 394b) may permit the user to direct the tip 322 more precisely in conjunction with the desired target area and/or in order to direct the catheter 300 according to bends or junctions in the vasculature.

As shown in FIG. 3A, catheter body 300 can have or define a central longitudinal axis 302. Laser catheter system 30 can also include a guidewire 390, and a handle 366 for moving the guidewire 390 relative to the catheter 300. In some embodiments, the guidewire is a standard commonly available guidewire. The laser catheter system 30 may further include a proximal guidewire port 31 that is adapted to the receive the guidewire 390 or a mandrel, or both. The catheter 300 is comprised of, or is adapted to house, a plurality of optical fibers 330 for transmission of laser energy. The optical fibers 330 are disposed within the catheter 300, surrounded by an outer jacket 340 of the catheter body, and extended toward distal section 320 or distal tip 322, as shown in cross-section 3A-A. The optical fibers 330 are disposed around or surround a lumen 355 inside of the catheter 300. Guidewire lumen 355 can have a size or diameter that is within a range from about 0.3 mm to about 0.7 mm. Catheter system 30 may also include or be coupled with a laser system 35 for supplying laser energy to the fiber optics 330.

As noted above, the laser catheter system 30 can also comprise a guidewire 390, which may include a proximal end or section 365 and a distal end or section 392. Guidewire 390 can include one or more bends near the distal end of the guidewire, and can be inserted into the guidewire lumen 355. For example, guidewire 390 can be bent in a more distal segment or end 392, so as to form a first bend 394a and a second bend 394b, as depicted in FIG. 3B. The distal tip 397 of guidewire 390 may be formed in any desired shape. For example, distal tip 397 may be formed in the shape of a ball. Often, the distal tip will not include a ball. In some cases, all or a portion of the guidewire may include a radiopaque material. Guidewire 390 may have a total length L along axis 361 that is within a range from about 100 cm to about 300 cm. Guidewire 390 may also have a tapered distal part 368 that spans or extends a distance D along axis 361 that is within a range from about 10 cm to about 40 cm. In some cases, the bent segment 393 spans or extends a distance Z along axis 361 that is within a range from about 0.1 cm to about 1.5 cm. A distal tip ball may include a radiopaque material. The length of the bent segment 393 may be varied as desired. For example, bent segment 393 can have a length within a range from about 0.1 cm to about 4 cm. In some embodiments, the distance T between bend 394b and the distal tip or end 397 of the guidewire is within a range from about 3 cm to about 10 cm. Bend 394a of guidewire distal segment 392 can provide an angle β between a central longitudinal axis 361 of the guidewire proximal portion 395 and a central longitudinal axis of bent segment 393. The angle β of the bend 394a may be varied from between about 1 and about 89 degrees, with about 45 degrees comprising one embodiment. Angle β can in some cases be defined as the angle between the bent segment 393 and the longitudinal axis 361 that corresponds to the more proximal segment 395 of the guidewire 390.

Guidewire 390 can be configured to induce or contribute to a bend 321 in the catheter body, as depicted in FIGS. 3C and 3D. A proximal portion of guidewire lumen 355 can be generally aligned with or substantially parallel to central axis 302 of the proximal portion of catheter body 300, and the distal portion of guidewire lumen 355 can be generally aligned with or substantially parallel to a central axis 303 of the distal portion of catheter body 300. In some embodiments, the guidewire 390 is insertable into the guidewire lumen 355, with the proximal end 395 of the guidewire 390 extending beyond the proximal end 310 of the laser catheter. When the catheter 300 is disposed over the guidewire 390, or when guidewire 390 is inserted into catheter 300, the guidewire 390 can be biased against an occlusion or a vessel wall so as to produce a bend 321 in the catheter distal section 320. Bend 321 can be associated with or defined by an angle α between the central longitudinal axis 302 of the catheter body 300 and a central longitudinal axis 303 of the deflected end segment or portion 324 of the catheter body which may be aligned with segment 393. Angle ce of bend 321 may be varied from between about 1 degree and about 89 degrees, with about 45 degrees comprising one embodiment. When the guidewire 390 is manipulated or biased, it can produce an offset or deflection of the tip 322 of the catheter 300. Movement of the guidewire 390 from the proximal end 395 of the guidewire, for example by a handle 366, can induce relative movement between the guidewire 390 and the catheter body 300 or can compel a portion of the guidewire to press against an occlusion or lumen wall, and thus cause the distal end 322 of the catheter body 300 to offset or deflect off of the central axis 302 at an angle α so as to cause the laser energy from the optical fibers 330 to move in an arc, as further illustrated in FIG. 6A. The offset or deflection provided by the guidewire 390 can permit the laser energy to reach an area that is larger than (e.g. 2 times larger) the diameter of the distal end 322 of the catheter body 300. As seen in FIG. 3B, the distal segment 392 of the guidewire may have a diameter that is smaller than the diameter of the more proximal segment 395 of the guidewire. In some cases, the diameter of the distal segment 392 or the distal end or tip 397 is within a range from about 0.2 mm to about 0.5 mm.

In use, the catheter 300 can be positioned in a subject, for example by insertion over a previously placed guidewire 390 or otherwise, in proximity to a stenosis or occlusion 370 in a vascular wall 375, as depicted in FIGS. 3C and 3D. The guidewire 390 is inserted into the lumen 355 of the catheter 300 such that the bent segment 393 of the guidewire 390 is disposed at or near the more distal section 320 of the catheter. In some embodiments, the proximal end 395 of the guidewire 390 may extend proximally beyond the proximal end 310 of the laser catheter 300. In some cases, guidewire 390 may include or be a mandrel. Laser energy is applied according to methods known to those of ordinary skill in the art. In accordance with some embodiments, during application of laser energy, the guidewire 390 is biased against the occlusion or interior lumen wall or otherwise manipulated such that the laser energy is directed at an angle γ from the longitudinal axis 302 of the unbent section or more proximal segment 310 of the catheter, as depicted in FIG. 1C, or at an angle Δ from the longitudinal axis 302 of the unbent section or more proximal segment 310 of the catheter, as depicted in FIG. 1D, thus “sweeping” the occlusion with laser energy over an area that is greater that the surface area of the distal end of the catheter. This placement of the laser energy is further illustrated in FIG. 1C-C, where the catheter tip position 380 is shown as slightly below of offset from the central axis 302 of the catheter body, and in FIG. 1D-D, where the catheter tip position 380 is shown as slightly below or offset from the central axis 302 of the catheter body. Optionally, manipulation of the guidewire may impart a lateral offset in the catheter body, such that the longitudinal alignment of the catheter body within the vessel or lumen does not change, but rather is offset from a first longitudinal alignment to a second longitudinal alignment, where both the first and second longitudinal alignments are generally parallel with or aligned to the longitudinal alignment of the vessel or lumen. In some embodiments, a central longitudinal axis of the laser energy can correspond to the central longitudinal axis 303 of the deflected end segment or portion 324 of the catheter body. One or more bends, for example bends 394a, 394b, or both, can be disposed near the distal end 320 of the catheter body 300 such that movement of guidewire 390 from the proximal end 395 of the guidewire 390 causes the distal end 320 of the catheter body to rotate or deflect off of the central axis 302 so as to cause the laser energy from the optical fibers to move in an arc or path that sweeps an area that is greater than the diameter of the distal end 320 of the catheter body 300.

Some embodiments of the present invention encompass a method for treating a region in a vessel. The method can include inserting a laser catheter 300 into a vessel 375. The laser catheter 300 can have a proximal end 310, a distal end 320, a central axis 302 which can correspond with the proximal end, and a guidewire lumen 355. The guidewire lumen 355 can be generally aligned with the central axis 302. The laser catheter 300 can also include a plurality of optical fibers 330 extending to the distal end 320. The method also includes inserting a guidewire 390 into the guidewire lumen 355. The guidewire 390 can have a distal end 392, a proximal end 395, and one or more bends 394a, 394b near the distal end 392. The guidewire 390 can be advanced distally or otherwise inserted into the guidewire lumen 355, or the catheter 300 can be advanced distally along the guidewire 390, until the bend 394a is at or near the distal end 320 of the catheter body 300. The method can further include rotating or manipulating the guidewire 390 to place the distal tip 322 of the catheter body 300 at a certain location 380 within the vessel 375, where the location 380 is offset from the central axis 302 of the catheter body. Additionally, the method includes providing laser energy to the optical fibers 330 to permit laser energy to project from the distal tip 322 of the catheter body 300 at the certain location 380. In some embodiments, the method includes continuously moving or manipulating the guidewire 390 to sweep the laser energy in an arc within the vessel. Optionally, the method may include inducing deflection in the distal end 320 of the catheter body 300 by rotating or translating the guidewire 390 relative to the catheter 300 and biasing the guidewire against the occlusion or vessel wall so as to move the catheter tip, or rotating or translating the catheter 300 relative to the guidewire 390 and biasing the guidewire against the occlusion nor vessel wall to as to move the catheter tip. The method may also include coupling the laser catheter 300 to a laser system 35 to supplying laser energy to the fiber optics 330. The optical fibers 330 can surround the guidewire lumen 355. The catheter body 300 can include a jacket 340 surrounding the optical fibers 330. The guidewire 390 can be inserted between the optical fibers 330. In some embodiments, the guidewire 390 is inserted through the catheter body 300 until the bend 394a in the guidewire 390 is within about 0 cm to about 5 cm of the distal end or tip 322 of the catheter body 300. The guidewire 390 can include or can be a mandrel. The distal end 320 of the catheter body can have a diameter that is in the range from about 0.6 mm to about 2.5 mm, and the laser energy can be swept to ablate an area that is at least about 2 times the diameter of the distal end of the catheter body. The bend 394a in the guidewire 390 can have an angle relative to the central axis 302 of the catheter body 300 that can be in the range from about 1 degree to about 89 degrees. A method embodiment may include introducing a guidewire 390 into the vessel 375, inserting the laser catheter 300 over the guidewire 390 using the guidewire lumen 355 to situate the laser catheter 300 within the vessel 375. The catheter body 300 may further include a guidewire lumen 355 extending between the proximal end 310 and the distal end 320, and the method may include inserting a guidewire 390 through the guidewire lumen 355 and introducing the laser catheter 300 into the vessel 375 using the guidewire 390. In some cases, the guidewire 390 includes a pair of bends, and the method includes inserting the guidewire 390 through the catheter body 300, or advancing the catheter body 300 along the guidewire 390, such that one bend extends beyond the distal tip 322 and another bend is at the distal tip 322. In some cases, the guidewire 390 includes a plurality of bends near the distal end 320, and the method includes applying laser energy to the optical fibers 330 while distally advancing the laser catheter 300 over the plurality of bends. Optionally, the guidewire can include or can be a mandrel.

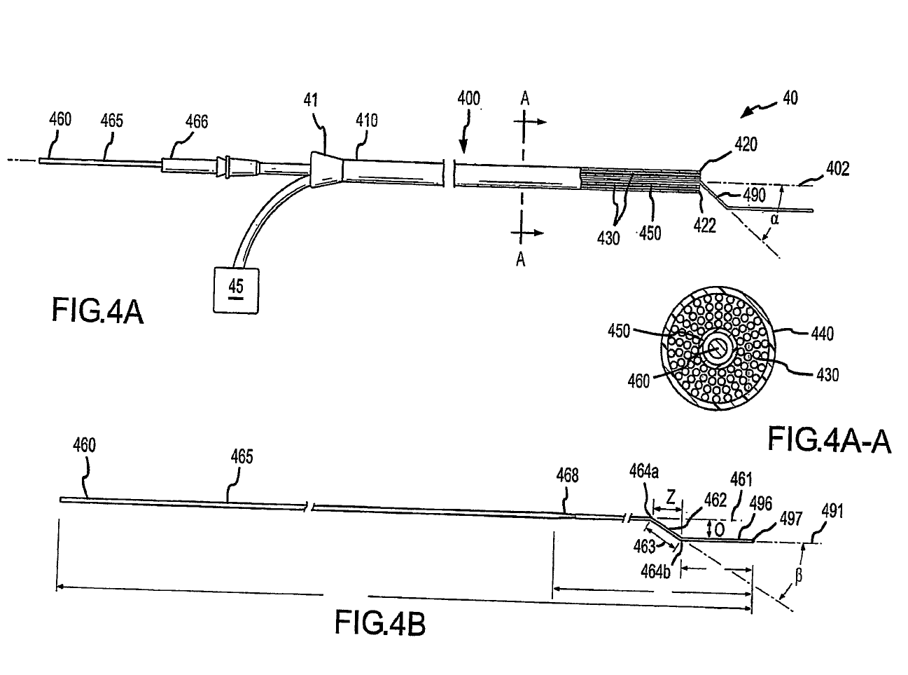

In an embodiment as depicted in FIGS. 4A-4B, a laser catheter system 40 comprises a laser catheter or catheter body 400 with a more proximal section 410 and a more distal section 420. The catheter 400 is comprised of a plurality of optical fibers 430 for transmission of laser energy that are disposed within the catheter 400 and surrounded by an outer jacket 440. The optical fibers 430 are disposed around a lumen 450 inside of the catheter 400. Without limiting the scope of the invention, the laser catheter system 40 also comprises a mandrel 460 that is bent in a more distal segment 462. In some embodiments, at about 3 cm to about 15 cm from a distal end or tip 497 of the mandrel 460, the mandrel 460 is bent at about a 30 degree offset at a more distal bend 464b, followed by another opposing more proximal bend 464a on the mandrel 460. The segment 463 of mandrel 460 between bends 464a and 464b can be any desired length, and can be considered to span a length Z along a central longitudinal axis 461 defined by the proximal section 465 of mandrel 460. The bends 464a, 464b are such that in use the distal tip 497 of the mandrel 460 can be offset from the longitudinal axis 402 of the catheter 400 a distance O which can be within a range from about 0.5 to about 2 mm when the catheter 400 is disposed over a more proximal portion 465 of the mandrel 460, as shown in FIG. 4B. When the catheter 400 is positioned in a subject in proximity to a stenosis or occlusion, the distal end 496 of the mandrel 460 is placed so as to penetrate or cross the occlusion. Laser energy may be applied according to methods known to those of ordinary skill in the art. In accordance with embodiments of the present invention, during application of laser energy, rotation of the bent mandrel 460 will produce deflection of the catheter tip 422 in a circular path, allowing the tip 422 to cover and ablate an area much larger than the diameter of the tip 422. The distal section 496 of the mandrel 460 can act as a strut to help obtain deflection of the catheter tip 422 within the artery. Thus, in accordance with embodiments of the present invention, the laser energy is directed by the rotation of the bent mandrel 460 at an angle from the longitudinal axis of the unbent section 465 of the catheter, “sweeping” the occlusion with laser energy over an area that is greater that the surface area of the distal end or tip 422 of the catheter 400. In addition, in some embodiments, without limitation, deflection of the tip 422 by one or more bends (e.g. bends 464a, 464b) may permit the user to direct the tip 422 more precisely in conjunction with the desired target area and/or in order to direct the catheter 400 according to bends or junctions in the vasculature.

In some cases, the distal tip of the mandrel may include a ball shape. In some cases, the distal tip of the mandrel may include a coil tip. For example, the mandrel may have a very small taper at the distal end, and a coil spring wrapped around or mounted on the small taper. Thus, the coil spring provides a larger distal diameter profile to the mandrel, and the mandrel maintains desired flexibility characteristics. The coil spring may include radiopaque materials that can be visualized under fluoroscopy. Optionally, the mandrel may include a plastic sleeve at the distal end of the mandrel, and the plastic sleeve may contain radiopaque filler.

As shown in FIG. 4A, catheter body 400 can have or define a central longitudinal axis 402. Laser catheter system 40 can also include a mandrel 460, and a handle 466 for rotating or translating the mandrel 460 relative to the catheter 400. The laser catheter system 40 may further include a proximal guidewire port 41 that is adapted to the receive the mandrel 460. The catheter 400 is comprised of, or is adapted to house, a plurality of optical fibers 430 for transmission of laser energy. The optical fibers 430 are disposed within the catheter 400, surrounded by an outer jacket 440 of the catheter body, and extended toward distal section 420 or distal tip 422, as shown in cross-section 4A-A. The optical fibers 430 are disposed around or surround a lumen 450 inside of the catheter 400. Mandrel lumen 450 can have a size or diameter that is within a range from about 0.2 mm to about 0.6 mm. Catheter system 40 may also include or be coupled with a laser system 45 for supplying laser energy to the fiber optics 430.

As noted above, the laser catheter system 40 can also comprise a mandrel 460, which may include a proximal end or section 465 and a distal end or section 462. Mandrel 460 can include one or more bends near the distal end of the mandrel, and can be inserted into the mandrel lumen 450. For example, mandrel 460 can be bent in a more distal segment or end 462, so as to form a first bend 464a and a second bend 464b, as depicted in FIG. 4B. The distal tip 497 of mandrel 460 may be formed in any desired shape. For example, distal tip 497 may be formed in the shape of a ball. In some cases, all or a portion of the guidewire may include a radiopaque material. Mandrel 460 may have a total length L along axis 461 that is within a range from about 100 cm to about 300 cm. Mandrel 460 may also have a tapered distal part 468 that spans or extends a distance D along axis 461 that is within a range from about 10 cm to about 40 cm. In some cases, the bent segment 463 spans or extends a distance Z along axis 461 that is within a range from about 0.1 cm to about 1.5 cm. A distal tip ball may include a radiopaque material. The length of the bent segment 463 may be varied as desired. For example, bent segment 463 can have a length within a range from about 0.1 cm to about 4 cm. In some embodiments, the distance T between bend 464b and the distal tip or end 497 of the mandrel is within a range from about 3 cm to about 10 cm. Bend 464a of mandrel distal segment 462 can provide an angle β between a central longitudinal axis 461 of the mandrel proximal portion 465 and a central longitudinal axis of bent segment 463. The angle β of the bend 464a may be varied from between about 1 and about 89 degrees, with about 45 degrees comprising one embodiment. Angle β can in some cases be defined as the angle between the bent segment 463 and the longitudinal axis 461 that corresponds to the more proximal segment 465 of the mandrel 460.

Mandrel 460 can be configured to induce or contribute to a bend in the catheter body. A proximal portion of mandrel lumen 450 can be generally aligned with or substantially parallel to central axis 402 of the proximal portion of catheter body 400, and the distal portion of mandrel lumen 450 can be generally aligned with or substantially parallel to a central axis of the distal portion of catheter body 400. In some embodiments, the mandrel 460 is insertable into the mandrel lumen 450, with the proximal end 465 of the mandrel 460 extending beyond the proximal end 410 of the laser catheter. When the catheter 400 is disposed over the mandrel 460, or when the mandrel 460 is inserted into catheter 400, the mandrel 460 can produce a bend in the catheter distal section 420. The bend can be associated with or defined by an angle α between the central longitudinal axis 402 of the catheter body 400 and a central longitudinal axis of the deflected end segment or portion of the catheter body which may be aligned with segment 463. Angle α of the bend may be varied from between about 1 degree and about 89 degrees, with about 45 degrees comprising one embodiment. When the mandrel 460 is rotated, it can produce circular deflection of the tip 422 of the catheter 400. Rotation of the mandrel 460 from the proximal end 465 of the mandrel, for example by a handle 466, can induce relative rotational movement between the mandrel 460 and the catheter body 400, and thus cause the distal end 422 of the catheter body 400 to rotate off of the central axis 402 at an angle α so as to cause the laser energy from the optical fibers 430 to move in an arc, as further illustrated in FIG. 6A. Accordingly, in some embodiments angle α corresponds with angle β. The bend 464a in the mandrel 460 can permit the laser energy to reach an area that is at least about 2 times the diameter of the distal end 422 of the catheter body 400. As seen in FIG. 4B, the distal segment 462 of the mandrel may have a diameter that is smaller than the diameter of the more proximal segment 465 of the mandrel. In some cases, the diameter of the distal segment 462 or the distal end or tip 497 is within a range from about 0.2 mm to about 0.5 mm.

In use, the catheter 400 can be positioned in a subject, for example by insertion over the mandrel 460 or otherwise, in proximity to a stenosis or occlusion in a vascular wall. The mandrel 460 can be inserted into the lumen 450 of the catheter 400, or the catheter 400 can be advanced over the mandrel 460, such that the bent segment 463 of the mandrel 460 is disposed at or near the more distal section 420 of the catheter. In some embodiments, the proximal end 465 of the mandrel 460 may extend proximally beyond the proximal end 410 of the laser catheter 400. In some cases, the mandrel 460 may include or be a guidewire. Laser energy is applied according to methods known to those of ordinary skill in the art. In accordance with some embodiments, during application of laser energy, the mandrel 460 is rotated or otherwise manipulated such that the laser energy is directed at an angle from the longitudinal axis 402 of the unbent section or more proximal segment 410 of the catheter, thus “sweeping” the occlusion with laser energy over an area that is greater that the surface area of the distal end of the catheter. This placement of the laser energy can be directed as desired. For example, the catheter tip position can be slightly below of offset from the central axis 402 of the catheter body. Optionally, the catheter tip position can be slightly below or offset from the central axis 402 of the catheter body. In some embodiments, a central longitudinal axis of the laser energy can correspond to the central longitudinal axis of the deflected end segment or portion of the catheter body, which can correspond to the central longitudinal axis of the mandrel segment 463. One or more bends, for example bends 464a, 464b, or both, can be disposed near the distal end 420 of the catheter body 400 such that rotation of mandrel 460 from the proximal end 465 of the mandrel 460 causes the distal end 420 of the catheter body to rotate off of the central axis 402 so as to cause the laser energy from the optical fibers to move in an arc that sweeps an area that is at least about 2 times the diameter of the distal end 420 of the catheter body 400.

Some embodiments of the present invention encompass a method for treating a region in a vessel. The method can include inserting a laser catheter 400 into a vessel. The laser catheter 400 can have a proximal end 410, a distal end 420, a central axis 402 which can correspond with the proximal end, and a mandrel lumen 450. The mandrel lumen 450 can be generally aligned with the central axis 402. The laser catheter 400 can also include a plurality of optical fibers 430 extending to the distal end 420. The method also includes inserting a mandrel 460 into the mandrel lumen 450. The mandrel 460 can have a distal end 462, a proximal end 465, and one or more bends 464a, 464b near the distal end 462. The mandrel 460 can be advanced distally or otherwise inserted into the mandrel lumen 450, or the catheter 400 can be advanced distally along the mandrel 460, until the bend 464a is at or near the distal end 420 of the catheter body 400. The method can further include rotating or manipulating the mandrel 460 to place the distal tip 422 of the catheter body 400 at a certain location within the vessel, where the location is offset from the central axis 402 of the catheter body. Additionally, the method includes providing laser energy to the optical fibers 430 to permit laser energy to project from the distal tip 422 of the catheter body 400 at the certain location. In some embodiments, the method includes continuously rotating or manipulating the mandrel 460 to sweep the laser energy in an arc within the vessel. Optionally, the method may include inducing deflection in the distal end 420 of the catheter body 400 by rotating or translating the mandrel 460 relative to the catheter 400, or rotating or translating the catheter 400 relative to the mandrel 460. The method may also include coupling the laser catheter 400 to a laser system 45 to supplying laser energy to the fiber optics 430. The optical fibers 430 can surround the mandrel lumen 450. The catheter body 400 can include a jacket 440 surrounding the optical fibers 430. The mandrel 460 can be inserted between the optical fibers 430. In some embodiments, the mandrel 460 is inserted through the catheter body 400 until the bend 464a in the mandrel 460 is within about 0 cm to about 5 cm of the distal end or tip 422 of the catheter body 400. The mandrel 460 can include or can be a guidewire. The distal end 420 of the catheter body can have a diameter that is in the range from about 0.6 mm to about 2.5 mm, and the laser energy can be swept to ablate an area that is at least about 2 times the diameter of the distal end of the catheter body. The bend 464a in the mandrel 460 can have an angle relative to the central axis 402 of the catheter body 400 that can be in the range from about 1 degree to about 89 degrees. A method embodiment may include introducing a mandrel 460 into the vessel, inserting the laser catheter 400 over the mandrel 460 using the mandrel lumen 450 to situate the laser catheter 400 within the vessel. The catheter body 400 may further include a mandrel lumen 450 extending between the proximal end 410 and the distal end 420, and the method may include inserting a mandrel 460 through the mandrel lumen 450 and introducing the laser catheter 400 into the vessel using the mandrel 460. In some cases, the mandrel 460 includes a pair of bends, and the method includes inserting the mandrel 460 through the catheter body 300, or advancing the catheter body 400 along the mandrel 460, such that one bend extends beyond the distal tip 422 and another bend is at the distal tip 422. In some cases, the mandrel 460 includes a plurality of bends near the distal end 420, and the method includes applying laser energy to the optical fibers 430 while distally advancing the laser catheter 400 over the plurality of bends. Optionally, the mandrel 460 can include or can be a guidewire.

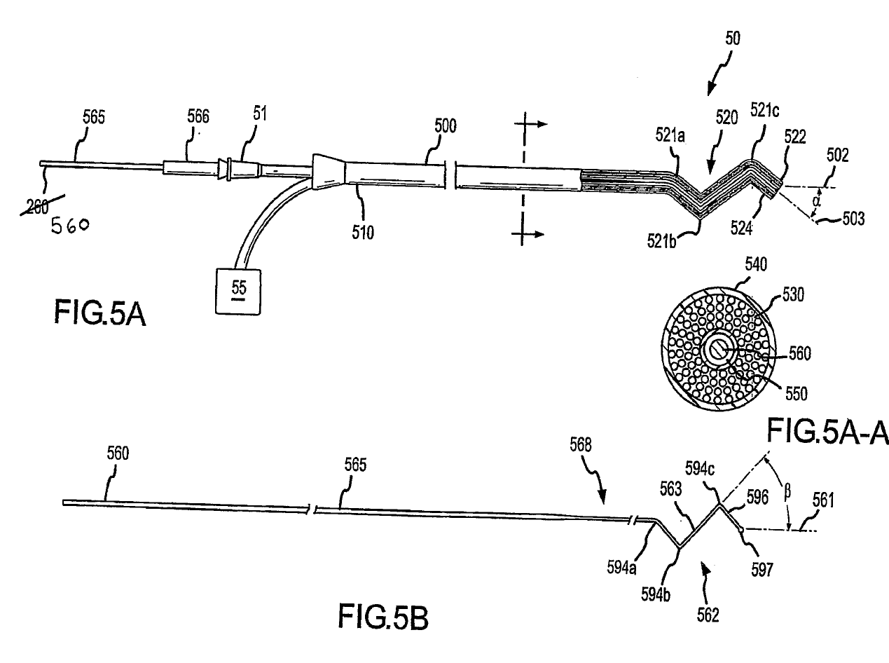

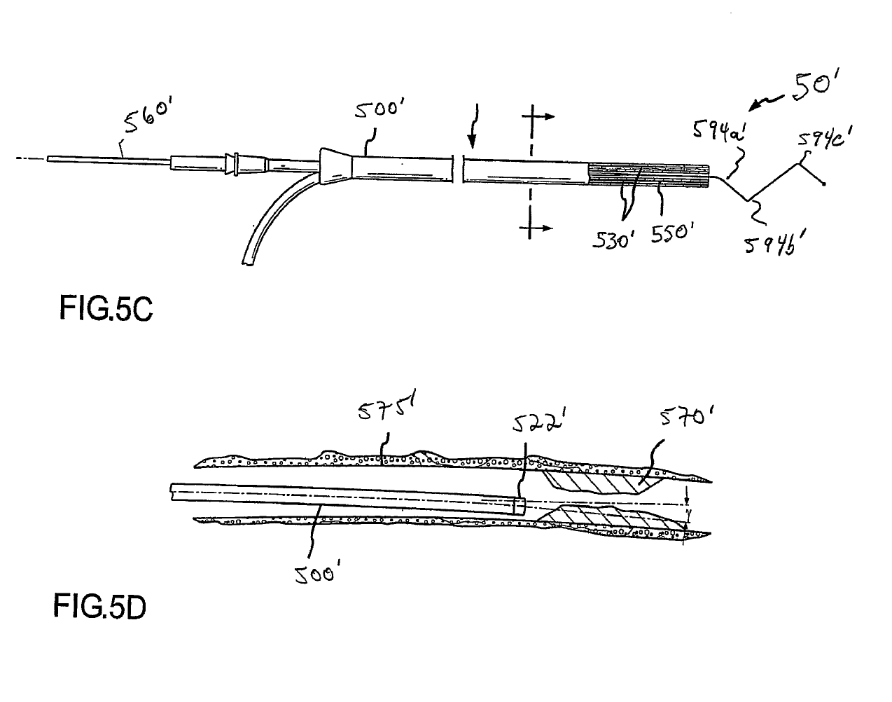

In an embodiment as depicted in FIGS. 5A-5B, a laser catheter system 50 comprises a laser catheter or catheter body 500 with a more proximal section 510 and a more distal section 520. The catheter 500 is comprised of a plurality of optical fibers 530 for transmission of laser energy that are disposed within the catheter 500 and surrounded by an outer jacket 540. The optical fibers 530 are disposed around a lumen 550 inside of the catheter 500. Without limiting the scope of the invention, the laser catheter system 50 also comprises a mandrel 560 that is bent in a more distal segment 562. In some embodiments, at about 3 to about 15 cm from a distal end or tip 597 of the mandrel 560, the mandrel 560 is bent at about a 30 degree offset at a more distal bend 594c, followed by another opposing more proximal bend 594b, and followed by still another even more proximal bend 594a. Mandrel 560 may include any number of such bends as desired. The bends 594a, 594b, and 594c are such that in use the distal tip 597 of the mandrel 560 is angularly offset from the central longitudinal axis 502 of the catheter 500. When the catheter 500 is positioned in a subject in proximity to a stenosis or occlusion, the distal end 596 of the mandrel 560 can be placed so as to penetrate or cross the occlusion. Laser energy may be applied according to methods known to those of ordinary skill in the art. In accordance with embodiments of the present invention, during application of laser energy, rotation or translation of the bent mandrel 560 can produce deflection of the catheter tip 522, allowing the tip 522 to cover and ablate an area much larger than the diameter of the tip 522. The distal section 596 of the mandrel 560 can act as a strut to help obtain deflection of the catheter tip 522 within the artery. Thus, in accordance with embodiments of the present invention, the laser energy is directed by the rotation or translation of the bent mandrel 560 at an angle from the longitudinal axis of the unbent section 510 of the catheter, “sweeping” the occlusion with laser energy over an area that is greater that the surface area of the distal end or tip 522 of the catheter 500. In addition, in some embodiments, without limitation, deflection of the tip 522 by one or more bends (e.g. bends 594a, 594b, 594c) may permit the user to direct the tip 522 more precisely in conjunction with the desired target area and/or in order to direct the catheter 500 according to bends or junctions in the vasculature.

As shown in FIG. 5A, catheter body 500 can have or define a central longitudinal axis 502. Laser catheter system 50 can also include a mandrel 560, and a torque or translation handle 566 for rotating or translating the mandrel 560 relative to the catheter 500. The laser catheter system 50 may further include a proximal guidewire port 51 that is adapted to the receive mandrel 560 or a guidewire, or both. The catheter 500 is comprised of, or is adapted to house, a plurality of optical fibers 530 for transmission of laser energy. The optical fibers 530 are disposed within the catheter 500, surrounded by an outer jacket 540 of the catheter body, and extended toward distal section 520 or distal tip 522, as shown in cross-section 5A-A. The optical fibers 530 are disposed around or surround a lumen 550 inside of the catheter 500. Mandrel lumen 550 can have a size or diameter that is within a range from about 0.2 mm to about 0.6 mm. Catheter system 50 may also include or be coupled with a laser system 55 for supplying laser energy to the fiber optics 530.

As noted above, the laser catheter system 50 can also comprise a mandrel 560, which may include a proximal end or section 565 and a distal end or section 562. Mandrel 560 can include one or more bends near the distal end of the mandrel, and can be inserted into the mandrel lumen 550. For example, mandrel 560 can be bent in a more distal segment or end 562, so as to form bends 564a, 564b, 564c, as depicted in FIG. 5B. The distal tip 597 of mandrel 560 may be formed in any desired shape. For example, distal tip 597 may be formed in the shape of a ball. In some cases, all or a portion of the mandrel may include a radiopaque material. Mandrel 560 may have a total length along axis 561 that is within a range from about 100 cm to about 170 cm. Mandrel 560 may also have a tapered distal part 568 that spans or extends a distance along axis 161 that is within a range from about 10 cm to about 40 cm. In some cases, the bent segment 563 spans or extends a distance along axis 561 that is within a range from about 0.1 cm to about 0.2 cm. A distal tip ball may include a radiopaque material. The length of the bent segment 563 may be varied as desired. For example, bent segment 563 can have a length within a range from about 0.1 cm to about 4 cm. In some embodiments, the distance between bend 594c and the distal tip or end 597 of the mandrel 560 is within a range from about 0.5 cm to about 2.5 cm. Bends 594a and 594b of mandrel distal segment 562 can provide an angle β between a central longitudinal axis 561 of the mandrel proximal portion 565 and a central longitudinal axis of bent segment 563. The angle β of the bend may be varied from between about 1 and about 89 degrees, with about 45 degrees comprising one embodiment. Angle β can in some cases be defined as the angle between the bent segment 163 and the longitudinal axis 161 that corresponds to the more proximal segment 165 of mandrel 560.

Mandrel 560 can be configured to induce or contribute to bends 521a, 521b, and 521c in the catheter body, as depicted in FIG. 5A. Thus, a proximal portion of mandrel lumen 550 can be generally aligned with or substantially parallel to central axis 502 of the proximal portion of catheter body 500, and the distal portion of mandrel lumen 550 can be generally aligned with or substantially parallel to a central axis 103 of the distal portion of catheter body 500. In some embodiments, the mandrel 560 is insertable into the mandrel lumen 550, with the proximal end 565 of the mandrel extending beyond the proximal end 510 of the laser catheter. When the catheter 500 is disposed over the mandrel 560, or when mandrel 560 is inserted or into catheter 500, the bent mandrel 560 produces a bend or bends in the catheter distal section 520. Bend 521c, for example, can be associated with or defined by an angle α between the central longitudinal axis 502 of the catheter body 500 and a central longitudinal axis 503 of the distal deflected end segment or portion 524 of the catheter body. Angle α may be varied from between about 1 degree and about 89 degrees, with about 45 degrees comprising one embodiment. When the mandrel 560 is rotated, it produces circular deflection of the tip 522 of the catheter 500. When mandrel 560 is translated, it produces transverse deflection of the tip 522 of the catheter 500. Rotation of the mandrel 560 from the proximal end 565 of the mandrel, for example by a torque handle 166, can induce relative rotational movement between the mandrel 560 and the catheter body 500, and thus cause the distal end 522 of the catheter body 500 to rotate off of the central axis 502 at an angle α so as to cause the laser energy from the optical fibers 530 to move in an arc, as further illustrated in FIG. 6A. Translation of the mandrel 560 from the proximal end 565 of the mandrel, for example by a translation handle 166, can induce relative translational movement between the mandrel 560 and the catheter body 500, and thus cause the distal end 522 of the catheter body 500 to deflect off of the central axis 502 so as to cause the laser energy from the optical fibers 530 to move in a transverse fashion, as further illustrated in FIG. 6B. The bends in the mandrel 560 can permit the laser energy to reach an area that is at least about 2 times the diameter of the distal end 522 of the catheter body 500. As seen in FIG. 5B, the distal segment 562 of mandrel may have a diameter that is smaller than the diameter of the more proximal segment 565 of the mandrel. In some cases, the diameter of the distal segment 562 or the distal end or tip 597 is within a range from about 0.2 mm to about 0.5 mm. In some embodiments, the catheter body is sufficiently stiff so as to resist deformation or bending when the bent mandrel is disposed therein. In some cases, even though the catheter body does not deflect or bend significantly, the catheter tip may deflect or bend slightly due to the presence of a mandrel bend at or near the catheter distal tip.

In use, the catheter 500 can be positioned in a subject, for example by insertion over a previously placed guidewire or otherwise, in proximity to a stenosis or occlusion in a vascular wall. The guidewire is removed, and/or the bent mandrel 560 is inserted into the lumen 550 of the catheter 500 such that a bent segment such as bent segment 563 of the mandrel 560 is disposed within the more distal section 520 of the catheter. In some embodiments, the proximal end 565 of the mandrel 560 may extend proximally beyond the proximal end 510 of the laser catheter 500. In some cases, mandrel 560 may include or be a guidewire. Laser energy is applied according to methods known to those of ordinary skill in the art. In accordance with some embodiments, during application of laser energy, the mandrel 560 is rotated such that the laser energy is directed at an angle α from the longitudinal axis 502 of the unbent section or more proximal segment 510 of the catheter, as depicted in FIG. 1B, thus “sweeping” the occlusion with laser energy over an area that is greater that the surface area of the distal end of the catheter. This placement of the laser energy can be directed as desired. For example, the catheter tip position can be slightly above or offset from the central axis 502 of the catheter body. Optionally, the catheter tip position can be slightly below or offset from the central axis 502 of the catheter body. In some embodiments, a central longitudinal axis of the laser energy can correspond to the central longitudinal axis 503 of the deflected end segment or portion 524 of the catheter body. A bend of a mandrel 560 can be disposed near the distal end 520 of the catheter body 500 such that rotation or translation of mandrel 560 from the proximal end 565 of the mandrel 560 causes the distal end 520 of the catheter body to rotate or traverse off of the central axis 502 so as to cause the laser energy from the optical fibers to move in an arc or path that sweeps an area that in some instances is at least about 2 times the diameter of the distal end 520 of the catheter body 500.

Some embodiments of the present invention encompass a method for treating a region in a vessel. The method can include inserting a laser catheter 500 into a vessel. The laser catheter 500 can have a proximal end 510, a distal end 520, a central axis 502 which can correspond with the proximal end, and a mandrel lumen 550. The mandrel lumen 550 can be generally aligned with the central axis 502. The laser catheter 500 can also include a plurality of optical fibers 530 extending to the distal end 520. The method also includes inserting a mandrel 560 into the mandrel lumen 550. The mandrel 560 can have a distal end 562, a proximal end 565, and one or more bends (e.g. bends 594a, 594b, 594c) near the distal end 562. The mandrel 560 can be advanced distally or otherwise inserted into the mandrel lumen 550 until one or more bends are at or near the distal end 520 of the catheter body 500. The method further includes rotating, translating, or otherwise manipulating the mandrel 560 to place the distal tip 522 of the catheter body 500 at a certain location within the vessel, where the location is offset from the central axis 502 of the catheter body. Additionally, the method includes providing laser energy to the optical fibers 530 to permit laser energy to project from the distal tip 522 of the catheter body 500 at the certain location. In some embodiments, the method includes continuously rotating or translating the mandrel 560 to sweep the laser energy in an arc or path within the vessel. Optionally, the method may include inducing deflection in the distal end 520 of the catheter body 500 by advancing the mandrel 560 into the mandrel lumen 550 or retracting the mandrel 560 proximally therefrom. Relatedly, deflection of the distal tip 522 of the catheter body 500 can be achieved by longitudinally translating the catheter body 500 relative to the mandrel 560, or by longitudinally translating the mandrel 560 relative to the catheter body 500, or both. The method may also include coupling the laser catheter 500 to a laser system 55 to supplying laser energy to the fiber optics 530. The optical fibers 530 can surround the mandrel lumen 550. The catheter body 500 can include a jacket 540 surrounding the optical fibers 530. The mandrel 560 can be inserted between the optical fibers 530. In some embodiments, the mandrel 560 is inserted through the catheter body 500 until one or more bends in the mandrel 560 are within about 0 cm to about 5 cm of the distal end or tip 522 of the catheter body 500. The mandrel 560 can include or can be a guidewire. The distal end 520 of the catheter body can have a diameter that is in the range from about 0.6 mm to about 2.5 mm, and the laser energy can be swept to ablate an area that is at least about 2 times the diameter of the distal end of the catheter body. One or more bends in the mandrel 560 can have an angle relative to the central axis 502 of the catheter body 500 that in the range from about 1 degree to about 89 degrees. Optionally, a method embodiment may include introducing a guidewire into the vessel, inserting the laser catheter 500 over the guidewire using the mandrel lumen 550 to situate the laser catheter 500 within the vessel and removing the guidewire prior to introducing the mandrel 560. The catheter body 500 may further include a guidewire lumen extending between the proximal end 510 and the distal end 520, and the method may include inserting a guidewire through the guidewire lumen and introducing the laser catheter 100 into the vessel 500 using the guidewire. In some cases, the mandrel 560 includes one or more bends, and the method includes inserting the mandrel 560 through the catheter body 500 such that one bend extends beyond the distal tip 522 and another bend is at the distal tip 522. In some cases, the mandrel 560 includes a plurality of bends near the distal end 520, and the method includes apply laser energy to the optical fibers 530 while distally advancing the laser catheter 500 over the plurality of bends. Optionally, the mandrel 560 can include or can be a guidewire.

As depicted in FIG. 5C, a laser catheter system 50′ can include a catheter body 500′ and a mandrel 560′ insertable therein. Catheter body 500′ includes optical fibers 530′ and a mandrel lumen 550′. Mandrel 560 includes a plurality of bends 594a′, 594b′, and 594c′. In use, the mandrel can be longitudinally translated within the mandrel lumen of the catheter body. The bends in the mandrel can impart an angular deflection γ in the catheter tip or an offset of the catheter body tip 522′ as shown in FIG. 5D. Accordingly, system 500 is well suited for ablating an occlusion 570′ in a vessel 575′. In use, the mandrel can be placed across an occlusion or lesion, and the catheter body can be placed or advanced over the mandrel. As the catheter body is advanced over the mandrel bends, the catheter will substantially straighten out the bends in the mandrel, however, the distal tip of the catheter will also be somewhat deflected as it passes over the bends.

FIGS. 6A and 6B illustrate aspects of exemplary systems and methods according to embodiments of the present invention. As discussed elsewhere herein, rotation of a mandrel, a guidewire, or a catheter can induce relative rotational movement between the mandrel or guidewire and the catheter body, and thus cause the distal end of the catheter body to rotate off of a central axis, such as a central longitudinal axis of a proximal or unbent portion of the catheter body, so as to cause the laser energy from the optical fibers to move in an arc or path. FIG. 6A shows such a “sweeping” technique for ablating an obstruction 670a contained within a vessel or lumen 675a of a patient. As the distal end of the catheter body rotates off of the central axis 602a to various catheter tip positions, for example catheter tip positions 680a(i), 680a(ii), and 680a(iii), laser energy can be directed along a central axis 603a so as to ablate or remove the occlusion. Often, the catheter tip sweeps from one tip position to the next in an arc or path 601a. This allows the laser energy to reach an area that is greater than the diameter of the distal end of the catheter body or the diameter of the distal end of the optical fibers. Similarly, as also discussed elsewhere herein, translation of a mandrel, a guidewire, or a catheter can induce relative translational movement between the mandrel or guidewire and the catheter body, and thus cause the distal end of the catheter body to traverse off of a central axis, such as a central longitudinal axis of a proximal or unbent portion of the catheter body, so as to cause the laser energy from the optical fibers to move in an path. FIG. 6B shows such a “sweeping” technique for ablating an obstruction 670b contained within a vessel or lumen 675b of a patient. As the distal end of the catheter body rotates off of the central axis 602b to various catheter tip positions, for example catheter tip positions 680b(i) and 680b(ii), laser energy can be directed along a central axis 603b so as to ablate or remove the occlusion. Often, the catheter tip sweeps from one tip position to the next in a path 601b. This allows the laser energy to reach an area that is greater than the diameter of the distal end of the catheter body or the diameter of the distal end of the optical fibers.

In some embodiments, an operator can simultaneously rotate a mandrel to deflect or sweep the catheter tip in a desired direction or path, advance the catheter in a body lumen, and ablate an obstruction with laser energy. In some cases, an operator may simultaneously advance the catheter in a body lumen and ablate an obstruction with laser energy, without rotating or deflecting the catheter tip. In some cases, an operator may perform a first discrete lasing step when the catheter tip is directed in a first position, then deflect, offset, or otherwise redirect the catheter tip, and subsequently perform a second discrete lasing step when the catheter tip is directed in the second position.

Optionally, guidewires or mandrels comprising the invention may be radiopaque, contain a radiopaque tip section, and/or contain one or more radiopaque markers so that the bent section can be positioned as desired during use. In some embodiments, without limitation, the distal tip of the guidewire or mandrel may comprise a rounded or ball shape. Rotary motion to the guidewire or mandrel may be applied manually and/or mechanically (as one example only and without limitation, by motorized torque device) to alleviate the user of the task and also provide more consistent motion of the catheter tip.

Guidewires and mandrels can transmit torque efficiently and rotate smoothly in order to transmit rotational deflection to the catheter tip. Guidewires and mandrels with a ground tapered core design are encompassed by the present disclosure, although all other types known to those of ordinary skill to be suitable also comprise embodiments of the invention. Bearing surfaces such as micro-coils, PTFE sleeves, PTFE coatings, and hydrophilic coatings are usable and can help provide smooth rotation.

In some embodiments, without limitation, the distal end of the bent mandrel may be approximately 0.007 inches in diameter. This diameter can easily penetrate a catheter inner lumen and therefore can be made blunt, as some examples only, by placing a solder ball on the end, forming a ball end by welding or welding on a radiopaque marker and rounding the end. Any of the structural or functional aspects of the guidewires described herein can be incorporated into or carried out by mandrels, and similarly any of the structural or functional aspects of the mandrels described herein can be incorporated into or carried out by the guidewires.

In accordance without with some embodiments, without limitation, it may desirable to bend the wire within the catheter remotely (from the proximal end) during the procedure. This may be accomplished by producing the wire from Ni/Ti and electrically actuating a material phase change to produce the bend. It may also be done by using pull wires or by using a sliding sleeve over the wire that holds the wire in a straight position normally, but when pulled back allows the wire to bend within the catheter.

The preceding description has been presented only to illustrate and describe exemplary embodiments of the methods and systems of the present invention. It is not intended to be exhaustive or to limit the invention to any precise form disclosed. The foregoing embodiments are illustrative, and no single feature or element is essential to all possible combinations that may be claimed in this or a later application. It will be understood by those skilled in the art that various changes may be made and equivalents may be substituted for elements thereof without departing from the scope of the invention. In addition, many modifications may be made to adapt a particular situation or material to the teachings of the invention without departing from the essential scope. Therefore, it is intended that the invention not be limited to the particular embodiment disclosed as the best mode contemplated for carrying out this invention. The invention may be practiced otherwise than is specifically explained and illustrated without departing from its spirit or scope. This description of the invention should be understood to include all novel and non-obvious combinations of elements described herein, and claims may be presented in a later application to any novel and non-obvious combination of these elements.

1. A laser catheter system, comprising:
a laser catheter comprising a catheter body having a proximal end, a distal end, a central axis and a mandrel lumen that is generally aligned with the central axis, wherein the laser catheter further includes a plurality of optical fibers extending to the distal end; and
a mandrel having a proximal end and a distal end, wherein the mandrel includes a bend near the distal end;
wherein the mandrel is insertable into the mandrel lumen, with the proximal end of the mandrel extending beyond the proximal end of the laser catheter, and the bend of the mandrel being near the distal end of the catheter body such that rotation of the mandrel from the proximal end of the mandrel causes the distal end of the catheter body to rotate off of the central axis so as to cause the laser energy from the topical fibers to move in an arc.

2. A system as in claim 1, further comprising a laser system for supplying laser energy to the fiber optics.

3. A system as in claim 1, wherein the mandrel comprises a guidewire.

4. A system as in claim 1, wherein the optical fibers surround the mandrel lumen and wherein the catheter body comprises a jacket surrounding the optical fibers.

5. A system as in claim 1, wherein the bend of the mandrel is within about 0.5 cm to about 2.5 cm of the distal end of the mandrel.

6. A system as in claim 1, wherein the distal end of the catheter body has a diameter that is in the range from about 0.5 mm to about 2.5 mm, and wherein the end in the mandrel permits the laser energy to reach an area that is at least about 2 times the diameter of the distal end of the catheter body.

7. A system as in claim 1, wherein the bend has an angle relative to the central axis that is in the range from about 1 degree to about 89 degrees.

8. A system as in claim 1, wherein the catheter body further includes a guidewire lumen extending between the proximal end and the distal end, and further comprising a guidewire that is insertable through the guidewire lumen.

9. A system as in claim 1, wherein the mandrel includes a plurality of bends near the distal end.

10. A system as in claim 1, wherein the mandrel has a diameter near the distal end that is in the range from about 0.1 mm to about 0.5 mm and wherein the distal end is formed in the shape of a ball.

11. A laser catheter system, comprising:
a laser catheter comprising a catheter body having a proximal end, a distal end, a central axis and a mandrel lumen that is generally aligned with the central axis, wherein the mandrel lumen has a size in the range from about 0.2 mm to about 0.7 mm, wherein the laser catheter further includes a plurality of optional fibers extending to the distal end, wherein the distal end of the catheter body has a diameter that is in the range form about 0.5 mm to about 2.5 mm; and
a mandrel having a proximal end and a distal end, wherein the mandrel includes a bend near the distal end;
wherein the mandrel is insertable into the mandrel lumen, with the proximal end of the mandrel extending beyond the proximal end of the laser catheter, and the bend of the mandrel being near the distal end of the catheter body such that movement of the mandrel from the proximal end of the mandrel causes the distal end of the catheter body to move off of the central axis.

12. A system as in claim 11, wherein the mandrel comprises a guidewire.

13. A system as in claim 11, wherein the bend of the mandrel is near the distal end of the catheter body such that rotation of the mandrel from the proximal end of the mandrel causes the distal end of the catheter body to rotate off of the central axis.

14. A system as in claim 11, wherein movement of the distal end of the catheter body off of the central axis causes the laser energy from the optical fibers to move in a path that ablates an area that is at least about 2 times the diameter of the distal end of the catheter body.

15. A method for treating a region in a vessel, the method comprising:
inserting a laser catheter into a vessel, the laser catheter comprising a catheter body having a proximal end, a distal end, a distal tip at the distal end, a central axis and a mandrel lumen that is generally aligned with the central axis, wherein the laser catheter further includes a plurality of optical fibres extending to the distal end;
inserting a mandrel into the mandrel lumen, wherein the mandrel has a distal end, a proximal end and a bend near the distal end, wherein the mandrel is insert until the bend is near the distal end of the catheter body;
rotating the mandrel to place the distal tip of the catheter body at a certain location within the vessel which is offset from the central axis; and
providing laser energy to the optical fibers to permit laser energy to project from the distal tip at the certain location.

16. A method as in claim 15, further comprising continuously rotating the mandrel to sweep the laser energy in an arc within the vessel.

17. A method as in claim 15, further comprising coupling the laser catheter to a laser system to supplying laser energy to the fiber optics.

18. A method as in claim 15, wherein the optical fibers surround the mandrel lumen, wherein the catheter body comprises a jacket surrounding the optical fibers and wherein the mandrel is inserted between the optical fibers.

19. A method as in claim 15, wherein the mandrel is inserted through the catheter body until the bend in the mandrel is within about 0 cm to about 5 cm of the distal end of the catheter body.

20. A method as in claim 15, wherein the mandrel comprises a guidewire.

21. A method as in claim 15, wherein the distal end of the catheter body has a diameter that is in the range from about 0.6 mm to about 2.5 mm, and wherein laser energy is swept to ablate an area that is at least about 2 times the diameter of the distal end of the catheter body.

22. A method as in claim 15, wherein the bend has an angle relative to the central axis that is in the range form bout 1 degree to about 89 degrees.

23. A method as in claim 15, further comprising introducing a guidewire into the vessel, inserting the laser catheter over the guidewire using the mandrel lumen to situate the laser catheter within the vessel, and removing the guidewire prior to introducing the mandrel.

24. A method as in claim 15, wherein the catheter body further includes a guidewire lumen extending between the proximal end and the distal end, and further comprising inserting a guidewire through the guidewire lumen and introducing the laser catheter into the vessel using the guidewire.

25. A method as in claim 15, wherein the mandrel includes a pair of bends, and wherein the mandrel is inserted through the catheter body such that the first bend extends beyond the distal tip and the second bend is at the distal tip.

26. A method as in claim 15, wherein the mandrel includes a plurality of bends near the distal end and further comprising apply laser energy to the optical fibers while distally advancing the laser catheter over the plurality of bends.

27. A method as in claim 25, wherein the mandrel comprises a guidewire.
